(19) United States
(12) Patent Application Publication
(10) Pub. No.: US 2015/0174285 A1
(43) Pub. Date: Jun. 25, 2015

(54) SELF-SUPPORTING INTERFACE DRESSING

(71) Applicant: LABORATOIRES URGO, Chenove (FR)

(72) Inventors: Stephane Auguste, Ruffey Les Echirey (FR); Anne-Sophie Danerol, Dijon (FR); Aurelie Laborde, Marsannay La Cote (FR); Nadege Desmaison, Tart Le Haut (FR)

(73) Assignee: LABORATORIES URGO, Chenove (FR)

(21) Appl. No.: 14/413,387

(22) PCT Filed: Jul. 15, 2013
(86) PCT No.: PCT/FR2013/051690

§ 371 Date: Jan. 7, 2015

(30) Foreign Application Priority Data
Jul. 17, 2012 (FR) 1256909

(51) Int. Cl. A61L 15/44; A61L 15/24; A61L 15/34; A61L 15/50

(52) U.S. Cl. CPC A61L 15/44; A61L 15/50; A61L 15/24; A61L 15/34

(57) ABSTRACT

The present invention relates to a self-supporting interface dressing formed from a thin layer of a composition comprising a hydrophobic matrix and having through holes.

According to the invention, this hydrophobic matrix comprises:
per 100 parts by weight of a styrene-saturated olefin-styrene triblock copolymer having a viscosity between 0.2 and 2 Pa.s, as measured in a 10% (weight/weight) solution in toluene;
from 400 to 1220 parts by weight of a plasticizer, preferably a plasticizing oil; and
from 0 to 720 parts by weight of petroleum jelly;
   it being specified moreover that:
the total amount of plasticizer and petroleum jelly is greater than or equal to 750 parts by weight;
the amount of petroleum jelly is between 400 and 720 parts by weight when the amount of plasticizer is between 1000 and 1220 parts by weight.

Application: treatment of wounds

The present invention relates to a self-supporting (or support-free) interface dressing intended for the treatment of wounds.

Within the context of the treatment of wounds, dressings intended to be applied directly to the wound while providing an interface between said wound and an absorbent compress placed on said dressing in order to absorb the wound exudates have been used for many years. Such dressings are usually referred to as “interface dressings”.

Among the interface dressings currently on the market, mention may be made for example of the product sold by the company Laboratoires URGO under the name Urgotul®.

This product, which is in particular described in example 1 of document WO 00/16 725 generally consists of a reinforcement made of an open-mesh fabric, the yarns of which are coated with a cohesive and non-adherent gel in such a way as to leave the meshes essentially unfilled.

This gel is formed of a highly plasticized hydrophobic elastomeric matrix containing, as a dispersion, a small amount of hydrophilic particles of a hydrocolloid.

The Urgotul® dressing is particularly advantageous in so far as it does not adhere to the newly regenerated tissues and maintains optimal moisture conditions favorable for healing, while preventing the risk of wound maceration.

However, due to the stiffness of its reinforcement, the Urgotul® product lacks conformability and cannot easily be used for the treatment of wounds that are difficult to cover due to their location, such as for example the digits of the hand or the joints such as the elbow.

Within this context, it would be desirable to have an interface dressing that would exhibit the same advantages as the Urgotul® product but that would be free of reinforcement, that is to say self-supporting. Such a dressing will have to have a sufficient stiffness in order to be easily handled and applied to a wound.

However, the production of such a product, which must satisfy properties that are sometimes conflicting, is particularly complex.

Specifically, this product must be:
as thin as possible in order to be conformable, while having sufficient cohesion to be able to be handled without risk of tearing both during the manufacture thereof and during the use thereof;
sufficiently extensible in the cross direction and in the longitudinal direction, while remaining cohesive;
preferably transparent or translucent to enable care staff to monitor wound healing without removing the dressing;
easy to manufacture.

Moreover, this product must also retain the properties of non-adherence to the skin and to the wound, and the oily but pleasant feel of the Urgotul® product, and guarantee the promotion of the healing process and in particular fibroblast proliferation.

The compositions of the elastomeric matrices used in the manufacture of the Urgotul® dressing and of its current commercial variations do not make it possible to obtain a self-supporting, i.e. reinforcement-free dressing.

In French patent no. 2 936 158, a reinforcement-free interface dressing is proposed that is in the form of a thin layer comprising through holes that allow the flow of exudates and consisting of a specific hydrophobic matrix. This hydrophobic matrix comprises an elastomer consisting of a linear triblock/diblock copolymer of styrene and isoprene (SIS/SI), a plasticizing oil, a polyethylene and hydrocolloid particles in specific relative proportions.

The interface dressing described in this French patent no. 2 936 158 has good conformability properties, but this product is not completely satisfactory, especially due to the nature of its constituent matrix.

Specifically, this elastomeric matrix comprises a copolymer with an oxidation-sensitive unsaturated central block (isoprene), which may therefore be degraded over time thus adversely affecting the initial properties of the product with the concomitant risk of less effective healing.

This interface dressing has not been sold to date.

Thus, while there has long been a need to provide a product that is more conformable than the Urgotul® product, which has been sold for more than 10 years, no self-supporting interface dressing has been sold to date.

Within this context, it has been discovered, and this constitutes the basis of the present invention, that it was possible to develop a self-supporting dressing having the same advantages as the Urgotul® product, using elastomeric matrices having compositions much closer to that of the Urgotul® product than French patent no. 2 936 158 proposes.

In particular, the elastomeric matrices capable of being used for the development of the interface dressing according to the invention do not comprise an elastomer with an unsaturated central block, nor additional compounds, such as polyethylene, as is recommended in the abovementioned French patent. Therefore, these matrices are not liable to be degraded by oxidation over time and guarantee the maintenance of the initial properties of the dressing.

More specifically, the elastomeric matrices used within the context of the present invention comprise, as sole elastomer, a specific triblock copolymer of styrene-saturated olefin-styrene type which has a viscosity of between 0.2 and 2 Pa.s as measured in a 10% (weight/weight) solution in toluene, said elastomer being present within the elastomeric matrix in a predetermined amount by weight and in specifically selected relative proportions with the other components of the matrix (plasticizer and petroleum jelly).

Thus, according to a first aspect, one subject of the present invention is a self-supporting interface dressing formed from a thin layer of a composition comprising a hydrophobic matrix and having through holes, said hydrophobic matrix comprising:
per 100 parts by weight of a styrene-saturated olefin-styrene triblock copolymer having a viscosity between 0.2 and 2 Pa.s, as measured in a 10% (weight/weight) solution in toluene;
from 400 to 1220 parts by weight of a plasticizer, preferably a plasticizing oil; and
from 0 to 720 parts by weight of petroleum jelly;
   it being specified moreover that:
the total amount of plasticizer and petroleum jelly is greater than or equal to 750 parts by weight;
the amount of petroleum jelly is between 400 and 720 parts by weight when the amount of plasticizer is between 1000 and 1220 parts by weight.

According to one particular characteristic, the abovementioned hydrophobic matrix comprises, in addition, hydrocolloid particles in an amount of less than or equal to 25% by weight relative to the total weight of said hydrophobic matrix.

According to the invention, the abovementioned hydrophobic matrix generally consists of an elastomer, a plasticizer and optionally petroleum jelly.

This matrix comprises, as sole elastomer, a triblock block copolymer comprising two styrene thermoplastic end blocks and one elastomeric central block consisting of a saturated polyolefin.

This saturated polyolefin may be of poly(ethylene/butylene) or poly(ethylene/propylene) type.

Triblock copolymers having a saturated central block are well known to a person skilled in the art.

These compounds have various properties and differ, in particular, by their viscosity.

It has been discovered that the production of a self-supporting dressing is dependent on the choice of a copolymer of specific viscosity.

Thus, within the context of the present invention, the elastomer will be specifically selected from the copolymers of the type having a high molecular weight, characterized by a viscosity of between 0.2 and 2 Pa.s, as measured in a 10% (weight/weight) solution in toluene.

Triblock copolymers having a saturated central block that correspond to this property are for example sold:
by the company KRATON POLYMERS under the name KRATON® G1651 or KRATON® G1654 for the poly(styrene-ethylene/butylene-styrene) (abbreviated to SEBS) block copolymers;
by the company KURARAY under the name SEPTON® S2006 for the poly(styrene-ethylene/propylene-styrene) (abbreviated to SEPS) block copolymers and SEPTON® S8006 for the SEBS block copolymers.

Within the context of the present invention, SEBS or SEPS triblock copolymers having a styrene content of between 25% and 45% by weight, relative to the total weight of said copolymer, will be preferred.

Preferably, use will be made of the products sold by the company KRATON POLYMERS under the names KRATON® G1651 and KRATON® G1654.

In order to obtain a self-supporting interface dressing, not only must the nature of the elastomer used be specifically selected, but in addition this elastomer will have to be used in specific relative proportions within the hydrophobic matrix, as will be seen below.

The plasticizer used for the production of the hydrophobic matrix is intended to improve the stretching, flexibility, extrudability and processing properties of the abovementioned elastomer.

Preferably, this plasticizer will consist of a liquid or a mixture of liquids compatible with the saturated polyolefin central block of the elastomer used.

More preferably, this plasticizer is free of petroleum jelly and will be selected from the compounds that have a drop point of less than or equal to 35° C.

Among the plasticizers capable of being advantageously used, mention may be made in particular of plasticizing oils or else the synthetic products based on liquid mixtures of saturated hydrocarbons such as for example the products sold by the company TOTAL under the name GEMSEAL® and in particular the product GEMSEAL® 60 which is an isoparaffinic mixture resulting from a completely hydrogenated petroleum cut.

Within the context of the present invention, use will preferably be made of plasticizing oils, preferably plasticizing mineral oils and in particular mineral oils formed from compounds of paraffinic or naphthenic nature or mixtures thereof in variable proportions.

Particularly preferred plasticizing mineral oils are formed from mixtures of compounds of paraffinic and naphthenic nature, and in particular from such mixtures in which the proportion of compounds of paraffinic nature is predominant.

As examples of commercial plasticizing mineral oils, mention may be made of the products sold by the company SHELL under the name ONDINA®.

Among these products, excellent results have been obtained with the oils sold under the names ONDINA® 917 or ONDINA® 919.

As indicated above, the amount of plasticizer present within the hydrophobic matrix must be specifically selected as a function of the amount of elastomer.

Thus, per 100 parts by weight of elastomer, the hydrophobic matrix will contain from 400 to 1220 parts by weight, and preferably from 600 to 900 parts by weight, of plasticizer, preferably of a plasticizing oil.

In order to increase the oily feel and character of the mixture consisting of the elastomer and the plasticizer, it may be advantageous, within the context of the present invention, to add a predetermined amount of petroleum jelly to this mixture.

This will preferably be a commercially available petroleum jelly that conforms to the French pharmacopeia.

The amount of petroleum jelly within the hydrophobic matrix may be generally between 0 and 720 parts by weight, and preferably from 150 to 450 parts by weight, per 100 parts by weight of elastomer.

However, in order to obtain a self-supporting dressing, it has been observed that the following two conditions must inevitably be met:
the total amount of plasticizer and petroleum jelly must be greater than or equal to 750 parts by weight, per 100 parts by weight of elastomer;
the amount of petroleum jelly must be between 400 and 720 parts by weight when the amount of plasticizer is between 1000 and 1220 parts by weight.

The hydrophobic matrix that has just been described constitutes the essential element of the compositions that makes it possible to produce a self-supporting dressing in accordance with the invention.

Such compositions may nevertheless comprise additional compounds and in particular compounds selected from antioxidants, hydrocolloids and active agents or adjuvants commonly used in the field of wound treatment.

Thus, according to one particular embodiment, these compositions comprise one or more antioxidants.

Antioxidant or stabilizing compounds are commonly used to ensure the stability of the compounds incorporated into the formulation of the dressing compositions, in particular with respect to oxygen, heat, ozone or ultraviolet radiation.

As examples of antioxidants capable of being used within the context of the present invention, mention will especially be made of phenolic antioxidants, such as in particular the products sold by the company CIBA Specialty Chemicals under the names IRGANOX® and in particular the reference products IRGANOX® 1010, IRGANOX® 565 and IRGANOX® 1076.

Generally, these compounds will be able to be used in an amount of the order of from 0.05% to 1% by weight, preferably from 0.05% to 0.2% by weight, relative to the total weight of the composition.

Particularly advantageously, use will be made of the product IRGANOX® 1010 in an amount of between 0.05% and 0.1% by weight, relative to the total weight of the composition.

According to another embodiment of the invention that is particularly preferred within the context of wound healing, the compositions forming the dressing according to the invention comprise hydrophilic particles of a hydrocolloid.

The hydrocolloid particles are preferably dispersed homogeneously within the composition.

The term “hydrocolloid” or “hydrocolloid particles” is intended to denote here any compound normally used by a person skilled in the art for its ability to absorb aqueous liquids such as water, physiological saline or the exudates from a wound.

Within the context of the invention, these compounds will be used to promote a painless removal of the interface dressing and to maintain a moist environment at the wound in order to promote healing.

As suitable hydrocolloids, mention may be made, for example, of pectin, alginates, natural vegetable gums such as in particular gum karaya, cellulose derivatives such as carboxymethyl celluloses and their salts of an alkali metal such as sodium or calcium, and also the synthetic polymers based on acrylic acid salts, known under the name of “superabsorbents”, such as for example the products sold by the company BASF under the name LUQUASORB® 1003 or by the company CIBA Specialty Chemicals under the name SALCARE® SC91 and also the mixtures of these compounds.

The hydrocolloids which are preferred in the context of the present invention are the alkali metal salts of carboxymethyl cellulose, and in particular sodium carboxymethyl cellulose (CMC).

The size of the hydrocolloid particles is generally between 50 and 100 microns, in particular about 80 microns.

Superabsorbents described as “microcolloids” since they have a particle size of less than 10 micrometers may also be used.

The amount of hydrocolloid particles dispersed in the composition will generally be less than or equal to 25% and advantageously of the order of 2% to 20% by weight, preferably of 5% to 18% by weight, more preferably of 10% to 15% by weight relative to the total weight of the hydrophobic matrix.

The choice of an amount of hydrocolloid particles within these ranges of values is important for the production of a self-supporting interface dressing, in particular in order to prevent the gelling of the composition during the absorption of exudates from resulting in the closure of the through holes.

Particularly preferably, use will be made of a sufficiently small amount of hydrocolloid so that the interface dressing obtained has a limited absorption capacity, equivalent to that of the URGOTUL® product.

According to yet another embodiment, the compositions that make it possible to produce a self-supporting dressing according to the invention comprise one or more adjuvants and/or active agents commonly used in the field of wound treatment and more generally in the pharmacological field.

Such active agents are in particular substances that have a favorable role in the treatment of wounds and in particular are capable of inducing or accelerating healing during the wound cleaning and/or granulation phase.

The presence of hydrocolloids in the composition will favor the release of these active agents.

Among the other active agents capable of being used within the context of the invention, mention will be made, for example, of bactericidal or bacteriostatic agents, painkillers or local anesthetics and anti-inflammatories.

Generally, the compositions that form the dressings in accordance with the invention may comprise such active agents in an amount of from 0.01% to 20% by weight, preferably from 1% to 15% by weight and more preferably from 2% to 10% by weight, relative to the total weight of the composition.

Of course, the three particular embodiments that have just been described may be carried out separately or according to any combination thereof. The compositions that have just been described make it possible to produce self-supporting dressings.

For this purpose, these compositions will be formed into a thin layer with through holes, preferably positioned in a distributed manner in said layer.

The through holes may be made by perforation or punching of a composition previously formed into a thin layer, alone or combined with a temporary support or with a protective film customarily used for dressing manufacture, or else by a screened coating on a temporary support.

For further details, reference may be made to patent application FR 2 936 158.

Alternatively, the dressings in accordance with the invention may be manufactured by hot casting of a composition as described above on a plate engraved with the pattern used for forming the through holes, followed by cooling and demolding.

Generally, the dressings in accordance with the invention will have a thickness of between 0.4 mm and 2 mm, preferably of between 0.5 mm and 1 mm, more preferably of around 0.6 mm.

The through holes may be of any geometry and will have, for example, a circular, rectangular, trapezoidal or square cross section.

Their surface area will generally be between 0.25 and 5 mm².

These holes will in particular have a mean diameter of between 0.5 and 2 mm, preferably of the order of 1 mm, when their cross section is circular.

These holes will be distributed, preferably uniformly, with a density such that the total surface area of the holes represents between 20% and 70%, and preferably between 30% and 50% of the total surface area of the dressing.

According to one preferred embodiment, the self-supporting interface dressing according to the invention is in the form of a breathable net (or grid), preferably of square mesh having:
a thickness of the net of between 0.5 and 2 mm;
a “yarn width” (width of the space between two consecutive holes) of between 1 and 10 mm, and preferably of between 1 and 5 mm;
a grammage of between 200 and 1700 g/m², and preferably between 300 and 800 g/m².

According to one particularly preferred embodiment of the invention, such a dressing will be in the form of a square-mesh breathable net having:
a thickness of the net of 600 microns approximately;
a yarn width of the order of 2 mm;
a grammage of the order of 450 g/m².

Conventionally, such a dressing may be combined with a protective support deposited on each of its opposite faces in order to protect the dressing from the outside environment, said protectors being intended to be removed immediately before use.

Such protective supports are well known to a person skilled in the art and may for example consist of a siliconized polyester film.

Alternatively, and in order to further facilitate the handleability of the self-supporting interface dressing according to the invention, the latter could be combined with a protective system as described in application WO 2008/145 884 formed from a single sheet covering the two opposite faces of the dressing and facilitating the application thereof to the wound.

The invention will be illustrated by the following non-limiting examples.

### EXAMPLES 1 TO 3

### Preparation of a Self-Supporting Interface Dressing According to the Invention

The plasticizer and the hydrocolloid are introduced successively, with stirring, into a Z-arm kneader at a setpoint temperature of 90° C. and are kneaded until a homogeneous mixture is obtained.

After having brought the setpoint temperature to 140° C., the elastomer and an antioxidant are introduced, with stirring, and then kneaded until a homogeneous mixture is obtained.

The petroleum jelly is then introduced, with stirring, at 140° C. in two portions until a homogeneous mixture is obtained.

The mixture thus obtained is hot cast at a temperature of the order of 120-130° C. onto an engraved flat plate forming for example the imprint of a square-mesh net or grid.

After cooling and demolding, the expected dressing is obtained in the form of a square-mesh net having a thickness of around 600 μm, a mesh size of the order of 2 mm, a thickness of the order of 0.6 mm and a grammage of the order of 450 g/m².

The dressings thus produced were placed between two 50 μm thick temporary protective films made of siliconized polyester.

The dressings from examples 1 to 3 were produced using the following constituents, in the proportions by weight mentioned in table 1:
elastomer: poly(styrene-ethylene/butylene-styrene) (abbreviated to SEBS) block copolymer;
   KRATON® G1654 (example 1);
   KRATON® G1651 (examples 2 and 3)
Plasticizer: Ondina® 917 mineral oil sold by the company SHELL
antioxidant: IRGANOX® 1010 sold by the company CIBA Specialty Chemicals
petroleum jelly: Codex A petroleum jelly sold by the company AIGLON
hydrocolloid: sodium carboxymethyl cellulose: CMC BLANOSE® 7H4XF sold by the company ASHLAND.

TABLE I

| | | EXAMPLE 1 | | EXAMPLE 2 | | EXAMPLE 3 | |
|---|---|---|---|---|---|---|---|
| COMPOSITION | | % | Parts | % | Parts | % | Parts |
| ELASTOMER | KRATON G 1651 | | | 4.9 | 100.00 | 4.9 | 100.00 |
| | KRATONG 1654 | 8.3 | 100.00 | | | | |

| | | | | | | |
|---|---|---|---|---|---|---|
| PLASTICIZER | 61.5 | 740.96 | 55 | 1122.45 | 45 | 918.37 |
| PETROLEUM JELLY | 15 | 180.72 | 25 | 510.20 | 35 | 714.29 |
| ANTIOXIDANT | 0.2 | 2.41 | 0.1 | 2.04 | 0.1 | 2.04 |
| HYDROCOLLOID | 15 | 180.72 | 15 | 306.12 | 15 | 306.12 |
| | 100 | 1204.82 | 100 | 2040.82 | 100 | 2040.82 |

1. A self-supporting interface dressing formed from a thin layer of a composition comprising a hydrophobic matrix and having through holes, wherein said hydrophobic matrix comprises:
per 100 parts by weight of a styrene-saturated olefin-styrene triblock copolymer having a viscosity between 0.2 and 2 Pa.s, as measured in a 10% (weight/weight) solution in toluene;
from 400 to 1220 parts by weight of a plasticizer, and
from 0 to 720 parts by weight of petroleum jelly;
it being specified moreover that:
the total amount of plasticizer and petroleum jelly is greater than or equal to 750 parts by weight;
the amount of petroleum jelly is between 400 and 720 parts by weight when the amount of plasticizer is between 1000 and 1220 parts by weight.

2. The dressing as claimed in claim 1, wherein the abovementioned hydrophobic matrix comprises, per 100 parts by weight of the abovementioned copolymer:
from 600 to 900 parts by weight of a plasticizer, and
from 150 to 450 parts by weight of petroleum jelly.

3. The dressing as claimed in claim 1, wherein the abovementioned composition comprises, in addition, hydrocolloid particles in an amount of less than or equal to 25% by weight relative to the total weight of said hydrophobic matrix.

4. The dressing as claimed in claim 1, wherein the abovementioned composition comprises, in addition, one or more substances selected from substances that have a favorable role in the treatment of wounds, bactericidal or bacteriostatic agents, painkillers or local anesthetics, and also anti-inflammatories, in an amount of between 0.01 and 20% by weight, relative to the total weight of the composition.

5. The dressing as claimed in claim 1, wherein the abovementioned composition comprises, in addition, one or more antioxidants in an amount of from 0.05% to 1% by weight, relative to the total weight of the composition.

6. The dressing as claimed in claim 1, characterized in that it is in the form of a breathable net or grid having a thickness of between 0.4 and 2 mm.

7. The dressing as claimed in claim 1, which comprises through holes, it being possible for each hole to be of any geometry and the surface area of which is between 0.25 and 5 mm²; the total surface area of said holes representing between 20% and 70%, of the total surface area of the dressing.

8. The dressing as claimed in claim 1, wherein said plasticizer is a plasticizing oil.

9. The dressing as claimed in claim 2, wherein said plasticizer is a plasticizing oil.

10. The dressing as claimed in claim 8, wherein the abovementioned composition comprises, in addition, hydrocolloid particles in an amount of less than or equal to 25% by weight relative to the total weight of said hydrophobic matrix.

11. The dressing as claimed in claim 1, wherein the abovementioned composition comprises, in addition, one or more substances selected from substances that have a favorable role in the treatment of wounds, bactericidal or bacteriostatic agents, painkillers or local anesthetics, and also anti-inflammatories, in an amount of between 1 and 15% by weight, relative to the total weight of the composition.

12. The dressing as claimed in claim 8, wherein the abovementioned composition comprises, in addition, one or more substances selected from substances that have a favorable role in the treatment of wounds, bactericidal or bacteriostatic agents, painkillers or local anesthetics, and also anti-inflammatories, in an amount of between 0.01 and 20% by weight, relative to the total weight of the composition.

13. The dressing as claimed in claim 2, wherein the abovementioned composition comprises, in addition, one or more substances selected from substances that have a favorable role in the treatment of wounds, bactericidal or bacteriostatic agents, painkillers or local anesthetics, and also anti-inflammatories, in an amount of between 0.01 and 20% by weight, relative to the total weight of the composition.

14. The dressing as claimed in claim 1, wherein the abovementioned composition comprises, in addition, one or more antioxidants in an amount of from 0.05% to 0.2% by weight, relative to the total weight of the composition.

15. The dressing as claimed in claim 8, wherein the abovementioned composition comprises, in addition, one or more antioxidants in an amount of from 0.05% to 1% by weight, relative to the total weight of the composition.

16. The dressing as claimed in claim 2, wherein the abovementioned composition comprises, in addition, one or more antioxidants in an amount of from 0.05% to 1% by weight, relative to the total weight of the composition.

17. The dressing as claimed in claim 8, wherein it is in the form of a breathable net or grid having a thickness of between 0.4 and 2 mm.

18. The dressing as claimed claim 1, which comprises through holes, it being possible for each hole to be of any geometry and the surface area of which is between 0.25 and 5 mm²; the total surface area of said holes representing between 30% and 50% of the total surface area of the dressing.

19. The dressing as claimed claim 8, which comprises through holes, it being possible for each hole to be of any geometry and the surface area of which is between 0.25 and 5 mm²; the total surface area of said holes representing between 20% and 70% of the total surface area of the dressing.

20. A self-supporting interface dressing formed from a thin layer of a composition comprising a hydrophobic matrix and having through holes, wherein said hydrophobic matrix comprises:
per 100 parts by weight of a styrene-saturated olefin-styrene triblock copolymer having a viscosity between 0.2 and 2 Pa.s, as measured in a 10% (weight/weight) solution in toluene;
from 600 to 900 parts by weight of a plasticizer, and
from 150 to 450 parts by weight of petroleum jelly;
it being specified moreover that:
the total amount of plasticizer and petroleum jelly is greater than or equal to 750 parts by weight;
the amount of petroleum jelly is between 400 and 720 parts by weight when the amount of plasticizer is between 1000 and 1220 parts by weight;
said composition further comprises, in addition,
hydrocolloid particles in an amount of less than or equal to 25% by weight relative to the total weight of said hydrophobic matrix;
one or more substances selected from substances that have a favorable role in the treatment of wounds, bactericidal or bacteriostatic agents, painkillers or local anesthetics, and also anti-inflammatories, in an amount of between 0.01 and 20% by weight, relative to the total weight of the composition;
one or more antioxidants in an amount of from 0.05% to 1% by weight, relative to the total weight of the composition.
